(19) United States
(12) Patent Application Publication
(10) Pub. No.: US 2007/0142473 A1
(43) Pub. Date: Jun. 21, 2007

(54) DOSAGE FORMS AND METHODS USING ETHACRYNIC ACID

(76) Inventors: Lawrence Solomon, Boca Raton, FL (US); Allan S. Kaplan, Boca Raton, FL (US)

Correspondence Address: ACCU-BREAK TECHNOLOGIES, INC., 1000 SOUTH PINE ISLAND ROAD, SUITE 230, PLANTATION, FL, 33324, US

(21) Appl. No.: 11/611,454

(22) Filed: Dec. 15, 2006

Related U.S. Application Data
(60) Provisional application No. 60/750,769, filed on Dec. 15, 2005.

(51) Int. Cl. A61K 31/192

(52) U.S. Cl. 514/571

(57) ABSTRACT

The invention is directed to a method of treating systemic arterial hypertension which preferably uses enter administration of a once-daily dose of ethacrynic acid of less than 50 mg/day to a patient afflicted with arterial hypertension. Doses of less than 25 mg/day ethacrynic acid are also indicated for treating hypertension. The invention also includes novel dosage forms comprising from 2.0 to 20 mg of ethacrynic acid.

### CROSS-REFERENCE TO RELATED APPLICATIONS

This is a continuation-in-part of U.S. Provisional Patent Application, Ser. No. 60/750,769, filed Dec. 15, 2005.

### BACKGROUND OF THE INVENTION

Ethacrynic acid, currently marketed under the Merck & Co. trade name Edecrin®, was developed as a loop (“high-ceiling”) diuretic and was found to be effective to treat fluid retention and, at relatively high doses, hypertension. As used herein, “hypertension” means elevated blood pressure in the systemic arterial circulation (as opposed to pulmonary arterial hypertension, portal hypertension, etc.). The disclosed doses of ethacrynic acid for use in the treatment of hypertension are in the range of 25 mg/day to at least 200 mg/day, though only small-scale studies have been performed, and no evidence of efficacy under 50 mg twice daily has been experimentally found.

A 25 mg starting dose of ethacrynic acid was used to initiate treatment of edema of various causes or to prevent return of edema after successful treatment thereof. Less than daily treatment with ethacrynic acid has also been disclosed for edema treatment or prevention but has not been disclosed for hypertension, and only twice daily treatment for hypertension has been disclosed.

No indication from the Food and Drug Administration (“FDA”) or other regulatory agency has been obtained for the treatment of hypertension using ethacrynic acid. The prescribing information (“label”) for Edecrin states: “The safety and efficacy of Ethacrynic acid in hypertension have not been established.” Several small studies in the 1960's and 1970's suggested that 50 mg twice daily and greater doses of ethacrynic acid had some degree of efficacy in treating hypertension. Due to such factors as side effects and greater effectiveness of other diuretics in treating hypertension, ethacrynic acid has been neglected regarding a potential role in treatment of hypertension.

U.S. Pat. No. 6,000,328 lists ethacrynic acid as one of four loop diuretics that could be used to treat hypertension, and indicates its dosage at 25-100 mg/day, twice a day (BID). The lowest individual dose of ethacrynic acid speculated in the '828 patent is therefore 12.5 mg and, being administered BID, the lowest daily dose contemplated is 25 mg. No data based on clinical trials has been found by the inventors that supports the safety or efficacy of ethacrynic acid at a daily dose below 100 mg.

In U.S. Pat. Nos. 5,948,799 and 6,245,787, ethacrynic acid is disclosed as being used in the range of 12.5-400 mg daily. However, this use was limited to a very specific group of patients with congestive heart failure of non-ischemic etiology who were treated with amlodipine (the ′799 patent) or amlodipine plus an angiotensin converting-enzyme inhibitor (the ′787 patent).

It has been well demonstrated that a drug may have diuretic action but no predictable antihypertensive action. A current marketed example of this is Dyrenium® (triamterene), which is approved for edema reduction but not for hypertension. However, the product insert for Dyazide® (triamterene plus hydrochlorothiazide) states that triamterene has no predictable antihypertensive effect. Thus, use for ethacrynic acid as an inducer of salt and water excretion cannot per se predict antihypertensive effectiveness.

Experts in the field, in an article which detailed the strong salt and water-removing properties of ethacrynic acid (and the loop diuretic furosemide) stated: “The mechanism of the antihypertensive properties of these drugs is unclear . . . neither drug has been extensively utilized for chronic antihypertensive therapy because of the large dose requirements.”

Therefore, the lowest useful dose of ethacrynic acid that has been disclosed for hypertension is 25 mg/day when given BID. Although in U.S. Pat. No. 5,538,991, a dose as low as 5 mg/day of ethacrynic acid was speculated as being potentially useful for hypertension when used in conjunction with certain phenoxyphenylacetic acids having endothelin antagonist activity, such speculation depended on the mere possibility that said endothelin antagonist may potentiate any antihypertensive action of ethacrynic acid.

Ethacrynic acid is of special interest because all other FDA-approved antihypertensive diuretics are sulfonamide derivatives and are either contraindicated for use in sulfa-allergic patients or have a precaution against such use. The Edecrin Prescribing information provided by the U.S. marketer, Merck & Co., states that “the safety and efficacy of Edecrin in hypertension have not been established.”

It is believed that treating a hypertensive patient using ethacrynic acid at a dose of less than 50 mg per day on a once per day basis, preferably at about 2.5 to less than 25 mg/day in one or more divided doses, has not heretofore been demonstrated.

It is believed that treatment of hypertension with ethacrynic acid at a once-daily dose of about 50 mg per day or less has not been disclosed as safe and effective. It is also believed that treatment of hypertension with ethacrynic acid at a daily dose of about 20 mg or less has not been disclosed as safe and effective

### SUMMARY OF THE INVENTION

The subject invention provides a novel method of medically treating hypertension enterally, e.g. orally, using a once-daily dose of ethacrynic acid of about 50 mg/day, or less, preferably about 2.5 to about 25 mg daily, administered to a patient afflicted with arterial hypertension. The subject invention further provides a novel method of treating hypertension with a total daily dose of about 20 mg/day or less, dosed once or more than once daily. Preferable dosage ranges include from about 5.0 to about 12.5 mg or, more preferably, between about 7.5 to about 10 mg daily. It is contemplated that ethacrynic acid may be administered as monotherapy or in conjunction with one or more other antihypertensive drugs. The one or more antihypertensive drugs can be administered as part of the same or as part of a separate dosage form.

The invention provides a novel method of treating hypertension with a dose of ethacrynic acid consisting of, for example, from 10 to 25 mg once daily, either alone or in conjunction with another anti-hypertensive drug.

Accordingly, it is an object of the invention to provide a method of treating hypertension by administration of a low total daily dose of ethacrynic acid. Preferably, the total daily dose is less than 50 mg/day, administered once per day (QD), and more preferably, is less than 25 mg/day. Most preferred is administration of a low dose of ethacrynic acid to a sulfanomide-allergic patient suffering from hypertension.

It is another object of the invention to provide a method of treating hypertension that encompasses novel daily dosing of ethacrynic acid in the low (less than 25 mg/day) to very low (less than 15 mg/day) dosage range.

Another object of the subject invention concerns treating a hypertensive patient allergic or sensitive to sulfur-containing drugs by using a low or very low dose of ethacrynic acid.

A further use of ethacrynic acid in accordance with the subject invention is to treat hypertension, or edema or congestive heart failure in HIV positive patients. Preferably, the subject method can be useful to treat AIDS (acquired immunodeficiency disease) patients and “ARC” (AIDS-related complex) patients since these patients are known to have much higher rates of sulfonamide allergy than the general population.

These and other objects of the invention will become apparent from the appended specification.

### BRIEF DESCRIPTION OF THE DRAWINGS

FIG. 1A shows a cross-section of a taller than wide three-segment tablet according to an embodiment of the subject invention; FIG. 1B shows a cross-section of a tablet configured as the tablet shown in FIG. 1A, but having a score formed in the middle segment.

FIG. 2A is a cross-sectional view of a tablet shown in FIG. 1A, after the tablet has been broken through the unscored middle segment and yielding tablettes 10a and 10b, respectively; FIG. 2B is a front view of a tablet shown in FIG. 1B, after the tablet has been broken through the scored middle segment and yielding tablettes 11a and 11b, respectively.

FIG. 3 is an external perspective view of a three segment tablet comprising two unitary segments.

FIG. 4 is a cross-sectional view of the three segment tablet shown in FIG. 3.

FIG. 5 is a cross-sectional view of a tablet comprising two unitary segments, after the tablet has been broken through to yield tablettes 50 and 52.

FIG. 6 depicts a quadrisected tablet comprising four active segments contiguous with an inactive segment.

FIG. 7 depicts a cross-sectional view of a wider-than-tall. tablet comprising an inactive segment and a deeply scored segment.

FIG. 8 shows the tablet of FIG. 7 after it is broken through both segments to yield tablettes 70a and 70b.

### DETAILED DESCRIPTION OF THE INVENTION

In accordance with the subject invention, ethacrynic acid may be administered as a dosage form, formulated in conjunction with conventional excipients and tabletting aids which are welt known to those who are skilled in the art.

Administration of a preferred dosage form may be enter, but also may be by intravenous or transdermal routes, for example. Enteral administration is preferred to be oral, but may for example be through a feeding tube or as a suppository. Oral administration is preferred to be by use of a solid dosage form, such as a tablet or a capsule, but may for example be through a liquid.

The preferred daily enteral dose for treating hypertension in accordance with the subject method of treatment is less than 50 mg of ethacrynic acid administered once per day. More preferably, the dosage may be from about 2 mg to less than 50 mg daily, given as a single dose or as a divided dose or as a controlled release dosage form. Total daily doses of between 25 and 50 mg/day are preferably provided as a monotherapy (no other active drug provided as part of the treatment) and more preferably as a once per day dosing regimen.

In one preferred embodiment, an enteral dose of less than 25 mg/day, and more preferably between about 2.5 and about 25 mg daily, is administered to a patient afflicted with hypertension. The ethacrynic acid may, for example, be administered to a patient afflicted with hypertension at a dose of between 5 and 20 mg daily in at least two divided doses given at least six hours apart. In this method, twice daily dosing is preferred over more than twice daily dosing.

The invention also includes a pharmaceutical dosage form comprising about 2.0 to about 20 mg, preferably about 2.5 to about 12.5 mg, more preferably from about 5 to about 10 mg of ethacrynic acid in a tablet or capsule formulated with pharmaceutically acceptable excipients such as those described in Remington's Pharmaceutical Sciences 20^{th} Ed. (2000), which is incorporated by reference herein. The active and inactive pharmaceutical compositions useful in accordance with the subject invention comprise formulations that act as immediate-release or controlled-release compositions which include, but are not limited to, rapid-release, sustained release, delayed release, extended release, and the like, as readily understood by persons of ordinary skill in the pharmaceutical arts.

The ethacrynic acid may also be formulated as part of an accurately divisible dosage form. Useful embodiments of accurately divisible dosage forms are described, for example, in PCT/US2005/018631, PCT/US2005/018632, PCT/US2005/010633, PCT/US2005/018638, and PCT/US2005/018639, and their progeny, which are hereby incorporated by reference.

These referenced applications disclose embodiments of layered tablets wherein the layers are preferably configured to provide one or more active tablet segments and one or more inactive tablet segments as part of the same dosage form. Active segments comprise one or more active pharmaceutical ingredient (API); inactive segments are preferably substantially free of API, but may contain trace amounts or therapeutically inactive amounts of an API. The term “segment,” as used herein in describing the structure of a tablet, means the entirety of a contiguous, substantially homogeneous part of a tablet or tablette (see below) of the invention.

A segment comprising a single compressed layer is a “simple segment.” A “compound segment” is a segment formed from a plurality of adjacent layers each comprising a substantially identical composition. A layer is produced by introducing an amount of a single composition into a tablet die to fill at least a part of the die. A layer is considered to exist whether it is in the form of an un-tamped, tamped or fully compressed composition.

A preferred embodiment of the invention is a compressed pharmaceutical tablet having three segments—two end segments each comprising at Least one API, and a middle, inactive segment between the two active end segments. Preferably, the tablet is dimensioned to have a height that exceeds the width of said tablet, i.e. , the tablet is taller than it is wide. While one or all segments may individually have a width greater than height, the tablet as a whole has a height that exceeds its width.

The height of the tablet is measured vertically from the top to the bottom of the final, compressed tablet as it is positioned in the die. The width is measured as the greatest horizontal dimension of the tablet at a location halfway between the top and said bottom of said tablet, except that, when the horizontal cross-section of the tablet is substantially rectangular, then the width is defined as the length of a line perpendicular to the two shorter sides of the perimeter of the horizontal cross-section. The terms “vertical” and “horizontal” (“horizontal” may also be referred to as “transverse”) axes of the subject tablets are determined by, and have the same orientation as, the tablet die in which the tablet is compressed in a tablet press or other tabletting machine (“tablet press” herein) and the order of entry of granulations into the die.

Suitable dimensions for tablets according to this embodiment of the invention are: height: 6 mm to 24 mm; preferably 10 mm to 18 mm and more preferably from 10 mm to 14 mm; width (at the widest dimension of the horizontal axis): 2 mm to 16 mm; preferably 3 mm to 10 mm and more preferably 4 mm to 8 mm. Without limitation, the dimensions of the tablet may be optimal if the ratio of the height to the width is between about 1.5:1 to about 3:1.

Tablets of this embodiment of the subject invention are preferably produced on a layer press, such as a tri-layer or five-layer high speed press manufactured by Korsch AG of Germany. Remington's Pharmaceutical Sciences 20th Ed., Mack Publishing Co., Easton, Pa. (2000), Chapter 45, which is incorporated by reference, describes the various techniques utilized in making compressed tablets. Each segment can comprise a single layer of a composition (a simple segment), or each segment can comprise a plurality of layers (a compound segment).

As an example of a method of manufacture of a preferred tablet of the invention, first, a composition, e.g., a granulation, containing at least about one-half an effective dose of ethacrynic acid (“active granulation”) enters the die and is tamped to form a first segment. Second, a granulation substantially free of an API (an “inactive granulation”) enters the die and. As tamped. The inactive granulation creates a part of the tablet that can be identified and broken through so that a part of the drug containing a significant concentration of drug is not broken through. Last, a second active granulation enters the die, is optionally tamped, and then final compression occurs to form the third segment and the final compressed tablet. The third segment can comprise ethacrynic acid (e.g., in an amount to provide the remainder of the whole dose in the whole tablet) or can comprise a second API, different than ethacrynic acid. For example, one preferred embodiment of the subject “taller-than-wide” tablet comprises an effective dose of ethacrynic acid in a first end segment, and a second end segment comprising an effective dose of a potassium-retaining diuretic, an angiotensin converting enzyme (ACE) inhibitor, an angiotensin receptor blocker (ARB), an alpha blocker, a beta blocker, a calcium antagonist (calcium channel blocker), or an aldosterone antagonist. The first and second end segments are contiguous with and separated in the whole dosage form by a middle segment which preferably comprises a substantially drug-free, pharmaceutically acceptable composition.

It would be understood that each active end segment can also comprise a combination of APIs, preferably comprising ethacrynic acid with a second API selected from the group consisting of a thiazide-type diuretic, a potassium-retaining diuretic, an angiotensin converting enzyme (ACE) inhibitor, an angiotensin receptor blocker (ARB) an alpha blocker, a beta blocker, a calcium antagonist (calcium channel blocker), or an aldosterone antagonist.

A preferred combination includes ethacrynic acid and a second API which is compatible with, or that can be compatibly formulated with, ethacrynic acid. In addition, a third API can be included in either or both end segments, or can be formulated as part of the middle segment, thus having all three segments comprising an active composition. An API different than ethacrynic acid can also he included as part of the middle segment in embodiments comprising ethacrynic acid as the only API in each end segment, ethacrynic acid in a first end segment and a second API in the second end segment, or ethacrynic acid and a second API combined in the first and second end segments.

Subsequent to tablet formation, optionally, a score may be placed in the side of said tablet, preferably transversely. Alternatively, after tablet formation, a printed line or other forms of indicia such as dotted lines, symbols or perforations may be placed on or in the surface of the tablet, all of which serve the purpose of allowing identification of said tablet's desired breaking region from the standpoint of effecting accurate separation of the parts of a tablet containing isolated doses of drug. Other means of aiding identification of a region of potentially desired tablet breaking may be utilized such as the use of color to identify a particular segment.

In another embodiment of the subject invention, the ethacrynic acid API can be provided in a segmented tablet having “unitary segments” that each adjoin the same face (surface) of a compositionally distinct non-unitary segment. The term “unitary segment” means a physically separated, non-contiguous part of a divided layer of a tablet. A divided layer is preferably formed by employing an embossed die or punch in a tablet press, wherein the embossing causes the composition (e.g., granulation) to be divided into separate segments (though from the same layered composition) as it enters the tablet die or during compression. Alternatively, a divided layer can be formed by scoring of the tablet after final tablet compression, e.g., post-tabletting removal of a part of a segment to expose a contiguous non-unitary segment.

Tablets of this embodiment of the invention are also preferably produced as compressed tablets. A preferred machine with which to produce said tablets is a bi-layer, tri-layer, or five-layer tablet press (or, tabletting machine), as referred to above. The preferred embodiment is a tablet comprising a single inactive segment and a divided segment contiguous therewith, wherein the tablet is effectively formed as a bi-layer tablet. These bi-layer tablets of the subject invention are typically configured as tablets that are “wider-than-tall” in accordance with the descriptions provided herein.

In a preferred method of manufacturing a bi-layer embodiment of the subject invention, a first composition, e.g., a granulation, preferably containing a therapeutic amount of ethacrynic acid as the API enters an embossed tablet die and is tamped by an upper punch. Next, an inactive composition, is disposed onto the first composition, is preferably tamped by an upper punch, and then the tablet is compressed by the upper punch so that said compression pushes said first granulation below the highest points of said embossing. The embossing can comprise a pattern providing, for example, a bisecting or quadrisecting division of the first ethacrynic acid composition, and thus forming unitary active segments in a bisected or quadrisected pattern. The unitary segments formed in the tablet therefore allow precise division of said tablet, when desired, by allowing breaking to occur only through the segment comprising the inactive composition.

Another preferred embodiment of the invention involves a two segment tablet, as described above for the “unitary segment” embodiment, but wherein the embossing used in the formation of the active segment does not extend completely through the segment, but provides a “deep score” into the segment. A deep score means a score which extends at least about 50%, preferably greater than 70%, and more preferably greater than 90% into the segment. The embossing used to form the deeply scored segment is typically about 1 mm or greater in height, as measured from the immediately adjacent face of the die or punch. Preferably, the embossing is between about 1 mm and 3 mm, and more preferably between about 1 mm and 2 mm in height, forming a corresponding depth of score. This deep score embodiment, wherein the score extends up to, but not into, the inactive segment advantageously allows breaking through the tablet to provide tablettes that comprise a substantially predetermined quantity of active drug.

The preferred divisible tablet embodiments of the invention can be readily understood by reference to the drawings. The drawings depict vertical cross-sectional views (and one perspective view) of tablets and/or tablettes of the invention. Tablets are depicted as oriented in the die, so that the top of the tablet as shown corresponds with the top of the tablet in the die. In other words, the top segment of the tablet as viewed contains the last granulation to enter the die. Tablettes are depicted as oriented in the die before being separated from the intact tablet. Shaded (cross-hatched or stippled) areas represent active segments, i.e., those which contain an API or drug; clear (plain) areas represent inactive segments, i.e., segments comprising substantially drug-free compositions.

The cross-sectional view of a tablet that has a theoretical geometric plane passed through the tablet relative to a side which is arbitrarily designated as the front. Each front view represents a schematic cross-section that passes through the midpoint of the horizontal cross-section as measured from the front of the tablet to the back of the tablet or tablette. The front view is also parallel to the major axis of the tablet (e.g., for a tablet with a rectangular (but not square) transverse cross-section, the longer side of the perimeter is parallel with the plane that depicts the cross-sectional, front view). That plane is located half-way between the front and back surfaces of said tablet. Drawings are of tablets that have a rectangular but not square horizontal cross-section at the vertical mid-point of the tablet.

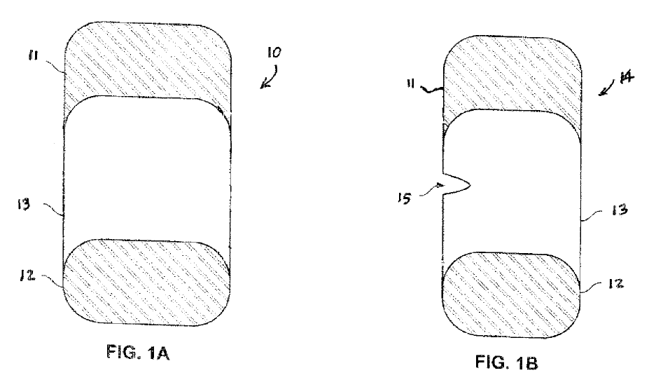

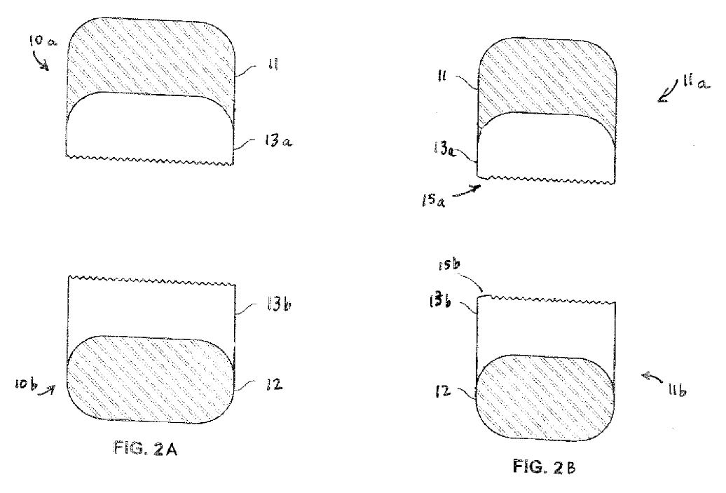

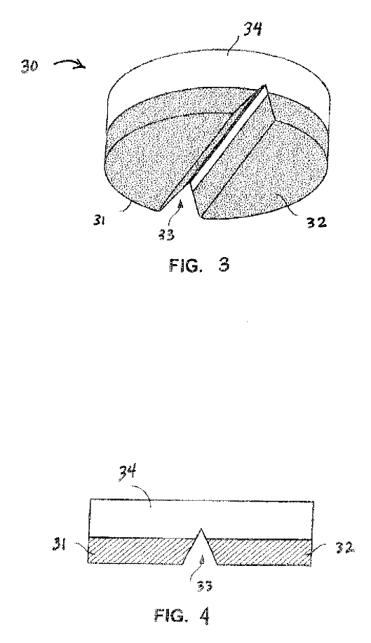

Tablettes are depicted with broken surfaces as indicated by a saw-tooth pattern. Such saw-tooth depiction is schematic and not intended to represent the actual pattern of breaking of a tablet (or tablette), which often leads to irregular edges even if said tablet is broken through a score. Tablets that are configured as taller-than-wide compositions are shown in FIGS. 1A, 1B, 2A, and 2B. Tablets configured as compositions that are wider-than-tall are shown in FIGS. 3, 4, 5 and 6.

FIG. 1A depicts a tablet 10 having a composition comprising an active pharmaceutical ingredient (API) in upper segment 11 and lower segment 12. In a preferred embodiment, each upper and lower active segment comprises a formulation which includes ethacrynic acid as the API. Inner segment 13 comprises a substantially drug-free (inactive) composition, but can contain trace amounts of the ethacrynic acid present in the upper and lower active segments. FIG. 2A depicts a tablet 14, substantially identical in composition and configuration to tablet 10 of FIG. 1A, but having a score 15 formed into the middle Inactive segment 13.

FIG. 2A depicts tablettes 10a and 10b formed from breaking tablet 10 of FIG. 1A, through its middle segment 13. In FIG.2A, unscored middle segments 13a and 13b are shown as broken through the approximate mid-line of the intact segment. FIG. 2B depicts tablettes 11a and 11b formed from breaking tablet 11 of FIG. 1B, through its middle segment 13. The middle segment 13 no longer exists as an intact segment and is shown in its divided form, comprising segment portions 13a and 13b. FIG. 2B, scored middle segments 13a and 13b are shown broken through the score in the intact segment. Score 15 of tablet 11 in FIG. 2B is shown divided into score parts 15a and 15b. Also, middle segment 13 no longer exists as an intact segment and is shown in its divided form, comprising segment portions 13a and 13b.

FIG. 3 is a perspective view of a bi-layer tablet 30 of the subject invention, having unitary segments 31 and 32, each comprising an active composition (containing an API), and preferably comprising ethacrynic acid. Segments 31 and 32 would be formed from the same layer or layers and creating score 33.

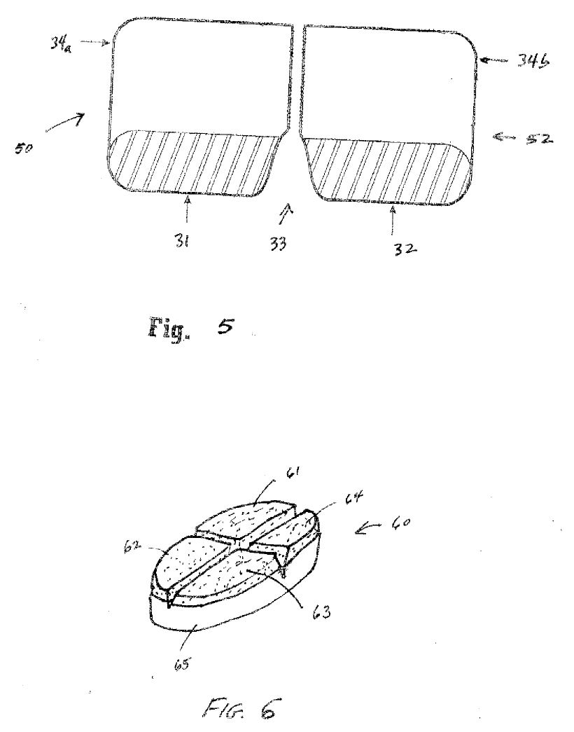

Score 33 can be formed by removing material from the bottom layer or layers of the active composition or by compressing one or more layers of the active composition in the presence of an embossing in the tablet die. To form separate segments 32 and 32, score 33 is completely through the active composition, exposing the overlying top segment 34 comprising an inactive composition. This inactive composition can advantageously support the integrity of the intact, whole tablet as well as the integrity of each segment, separately, after breaking of the tablet into its respective tablettes. A cross-sectional view of the bi-layer tablet of FIG. 3 is shown in FIG. 4, and illustrates the score that is formed completely through the layer(s) forming segments 31 and 32, and into inactive segment 34. FIG. 5 depicts the two tablettes 50 and 52 formed by breaking a tablet configured substantially the same as in FIG. 3 though score 33. Tablette 50 comprises segment 31 and inactive partial segment 34a; tablette 52 comprises intact segment 32 and inactive partial segment 34a. Segment 31 and segment 32 each remains intact after breaking of the whole tablet into its respective tablettes.

A quadrisected tablet, comprising four active segments 61, 62, 63, and 64, contiguous with inactive segment 65, is illustrated in FIG. 6.

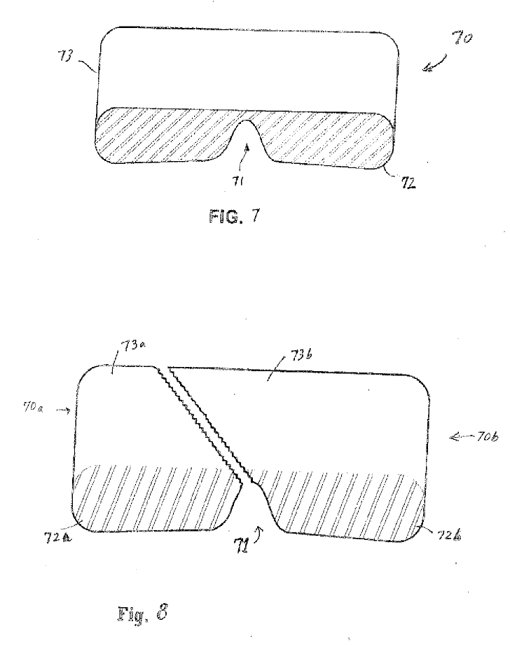

FIG. 7 depicts a tablet 70 having a deep score 71. In FIG. 7, the deep score 71 extends approximately 90% through the bottom segment 72. Upper segment 71 allows structural stability of the tablet despite the deep score 72. Breaking the tablet of FIG. 7 may gave two tablettes 70a and 70b, as shown in FIG. 8. Segment 72 is broken through score 71 into its respective portions, 72a and 72b; inactive segment 73 is broken into its respective portions 73a and 73b. Even though breaking as shown is far from vertical, it is clear that the amount of drug in new active segments 72a and 72b created from segment 72 (of FIG. 7) is substantially similar.

This description recites the use of ethacrynic acid as a preferred API in the tablets of the subject invention. However it is understood that the API can be any pharmaceutically acceptable drug. Moreover, it would be understood that the use of an API as described includes any pharmaceutically acceptable derivative, pro-drug, salt, polymorph, or isomer of that recited API.

When desired, ethacrynic acid may be administered in conjunction with one, two or three additional antihypertensive agents at doses that achieve the desired control of hypertensive conditions in particular patients. These additional anti-hypertensive agents which may be which may be co-administered as a separate dosage form or as part of a single combination dosage form, may for example comprise amlodipine when ethacrynic acid is used at doses lower than 25 mg/day, chlorthalidone (a thiazide-type diuretic), or one or more of the members of the group comprising a potassium-retaining diuretic, an angiotensin converting enzyme (ACE) inhibitor (e.g., benazepril, captopril, enalapril, lisinopril, perindopril, ramipril) an angiotensin receptor blocker (ARB) (e.g., losartan, valsartan, candesartan, eprosartan), or an alpha blocker (e.g., prazosin, doxazosin, terazosin); calcium antagonist (calcium channel blocker), e.g., amlodipine, nifedipine, nicardipine, nisoldipine, lacidipine, lercanidipine, manidipine, azelnidipine; aldosterone antagonist (e.g., spironolactone, canrenone, eplerenone; or beta blocker e.g. carvedilol, nebivolol, atenolol, metoprolol, betaxolol, bisoprolol) at appropriate doses such as the doses that are disclosed in the Physicians' Desk Reference (2005).

It is recognized that related inventions may be within the spirit of the disclosures herein. Also, no omission in the current application is intended to limit the inventors to the current claims or disclosures. While certain preferred and alternative embodiments of the invention have been set forth for purposes of disclosing the invention, modifications to the disclosed embodiments may occur to those who are skilled in the art.

1. A method of treating hypertension, said method comprising:
administering once per day a composition comprising ethacrynic acid as a monotherapy at a dose of no more than about 50 mg/day to a patient afflicted with hypertension.

2. The method of claim 1 wherein said treatment comprises administering ethacrynic acid at a dose of between 2.5 and 25 mg/day.

3. The method of claim 1 wherein said treatment comprises administering ethacrynic acid at a dose of about 20 mg/day.

4. A method of treating hypertension, said method comprising:
administering ethacrynic acid at a dose of less than 25 mg/day to a patient afflicted with hypertension.

5. The method of claim 4 wherein said treatment comprises administering ethacrynic acid at a dose of between 2.5 and 25 mg/day once per day.

6. The method of claim 4 wherein said treatment comprises administering ethacrynic acid at a dose of between 5 and 10 mg/day.

7. The method of claim 4 wherein administration of ethacrynic acid represents monotherapy for hypertension.

8. The method of claim 4 wherein said patient is not being treated with amlodipine.

9. The method of claim 4 wherein said treatment comprises administering ethacrynic acid and a second antihypertensive drug.

10. The method of claim 9 wherein treatment comprises administering a second antihypertensive drug and a third antihypertensive drug.

11. The method of claim 9 wherein said second antihypertensive drug is selected from the group consisting of a potassium-retaining diuretic, an angiotensin converting enzyme (ACE) inhibitor, an angiotensin receptor blocker (ARB), an alpha locker, an aldosterone antagonist, and a beta blocker.

12. The method of claim 4 wherein ethacrynic acid is administered at a dose of between 2.5 and 20 mg daily in at least two divided doses given at least six hours apart.

13. The method of claim 4 in which said method comprises administering ethacrynic acid at about 5-10 mg twice daily.

14. The method of claim 4 wherein said ethacrynic acid is administered at a total daily dose of between 2.5 and 10 mg/day.

15. The method of claim 12 wherein said divided doses are provided as an accurately divisible dosage form.

16. The method of claim 15 wherein said dosage form is a segmented tablet comprising at least one inactive segment.

17. The method of claim 15 wherein said dosage form comprises a score, said score provided in a scoring pattern selected from the group consisting of bisecting, trisecting, and quadrisecting scoring pattern.

18. A pharmaceutical dosage form indicated for treatment of hypertension, said dosage form comprising between 2.5 and 20 mg of ethacrynic acid.

19. The pharmaceutical dosage form of claim 18 comprising between 2.5 and 12.5 mg of ethacrynic acid.

20. The pharmaceutical dosage form of claim 18 comprising between 5 and 10 mg of ethacrynic acid.

21. The dosage form of claim 18 that is a solid pharmaceutical dosage form selected from the group consisting of a tablet and a capsule.

22. The pharmaceutical dosage form of claim 18 which consists essentially of ethacrynic acid and at least one pharmaceutical excipient.

23. The pharmaceutical dosage form of claim 18 further comprising a second antihypertensive drug.

24. The pharmaceutical dosage form of claim 23 further comprising a third antihypertensive drug.

25. The pharmaceutical dosage form of claim 18, said dosage form being indicated for once-a-day administration.

26. The pharmaceutical dosage form of claim 18, wherein said dosage form is a segmented tablet comprising at least one segment comprising a substantially pharmaceutically inactive composition.

27. The pharmaceutical dosage form of claim 26 wherein said segmented tablet has a height dimension and a width dimension wherein said height is greater than said width.

28. The pharmaceutical dosage form of claim 26 wherein said tablet comprises a score provided as a score pattern selected from the group consisting of bisecting, trisecting, and quadrisecting score pattern.

29. The pharmaceutical tablet of claim 28 wherein said score is a deep score.

30. A pharmaceutical tablet comprising a plurality of layers wherein at least one layer comprises a composition containing ethacrynic acid as an active ingredient.

31. The pharmaceutical tablet of claim 30 wherein said composition is an immediate release composition.

32. The pharmaceutical tablet of claim 30 wherein said tablet comprises at least one active segment comprising ethacrynic acid as an active ingredient and at least one inactive segment.

33. The pharmaceutical tablet of claim 32 wherein said tablet comprises two end segments comprising drug, said end segments positioned above and below a middle segment comprising a composition different than either of said two end segments.

34. The pharmaceutical tablet of claim 32 wherein said middle segment is substantially free of drug.

35. A pharmaceutical tablet comprising ethacrynic acid as an active ingredient, said tablet comprising a score selected from the group consisting of a bisecting score, a trisecting score, and a quadrisecting score.

36. A pharmaceutical tablet comprising about 20 mg or less ethacrynic acid, and a second active pharmaceutical ingredient.
